(19) United States
(12) Patent Application Publication
(10) Pub. No.: US 2025/0127654 A1
(43) Pub. Date: Apr. 24, 2025

(54) SYSTEMS AND METHODS FOR GENERATING A COLD SOLUTION

(71) Applicant: MIRAKI INNOVATION THINK TANK LLC, Cambridge, MA (US)

(72) Inventors: William Roger MAINWARING-BURTON, Cambridge, MA (US); George Robert Butcher, Cambridge, MA (US); Rainuka GUPTA, Newton, MA (US)

(73) Assignee: MIRAKI INNOVATION THINK TANK LLC, Cambridge, MA (US)

(21) Appl. No.: 18/688,213

(22) PCT Filed: Aug. 31, 2022
(86) PCT No.: PCT/US2022/042168

§ 371 Date: Feb. 29, 2024

Related U.S. Application Data
(60) Provisional application No. 63/238,952, filed on Aug. 31, 2021.

(51) Int. Cl. A61F 7/00; A61F 7/12

(52) U.S. Cl. CPC A61F 7/0085; A61F 2007/0063; A61F 2007/126

(57) ABSTRACT

Methods and systems for generating sterile cold solution are provided. The cold solution may be injected into subcutaneous fat of a subject to facilitate weight reduction or improve cosmetic appearances. The systems include a repository for receiving a solution, a generator for generating the cold solution from the solution, and a port for transferring the cold solution from the system. The generator includes a cooling device, a heat exchanger, and a recirculating fluid path configured to circulate a fluid which may include air. The methods include receiving a solution in the system, generating the cold solution from the solution, and transferring the cold solution from the system, wherein generating the cold solution includes circulating a fluid which may include air.

### TECHNICAL FIELD

The disclosure relates to systems and methods for generating a cold solution.

### BACKGROUND

Various medical and cosmetic benefits may be achieved through administration of a cold solution. In some cases, a cold solution may be administered via injection, and therefore must be sterile, able to flow through a delivery device such as a cannula or syringe, as well as possess other properties to achieve a desired medical or cosmetic benefit. Available systems and methods for generating a cold solution to achieve various medical and cosmetic benefits are unsatisfactory and therefore there is a need for such systems and methods.

### SUMMARY

The present invention provides systems and methods for generating a cold solution. The present invention provides:
(1) A system for generating a cold solution comprising a repository for receiving a solution; a generator for generating the cold solution from the solution; and a port for transferring the cold solution from the system; wherein the generator comprises a cooling device, a heat exchanger, and a recirculating fluid path configured to circulate a fluid.
(2) The system according to the above (1), wherein the fluid comprises air.
(3) The system according to the above (1), wherein the repository is insulated.
(4) The system according to the above (1), further comprising a valve configured to control flow.
(5) The system according to the above (1), further comprising a sterile loop configured for flow of the solution.
(6) The system according to the above (5), wherein the sterile loop interfaces with the fluid in the recirculating fluid path via the heat exchanger.
(7) The system according to the above (1), wherein the port is insulated.
(8) The system according to the above (1), wherein the port is configured for aseptic transfer.
(9) The system according to the above (8), further comprising a valve configured to control flow.
(10) The system according to the above (1), wherein the cooling device is configured as a cold sink.
(11) The system according to the above (10), wherein the cooling device is configured to operate at a temperature of about −10° C. to about −60° C.
(12) The system according to the above (10), wherein the cooling device is configured to operate at a temperature of about −10° C. to about −40° C.
(13) The system according to the above (1), wherein the cooling device comprises a single-phase coolant.
(14) The system according to the above (1), wherein the cooling device comprises a multi-phase refrigerant.
(15) The system according to the above (1), wherein the cooling device interfaces with the heat exchanger.
(16) The system according to the above (1), wherein the heat exchanger comprises a radiator.
(17) The system according to the above (16), wherein the heat exchanger interfaces with the cooling device via the radiator.
(18) The system according to the above (1), wherein the heat exchanger comprises an evaporator.
(19) The system according to the above (18), wherein the heat exchanger interfaces with the cooling device via the evaporator.
(20) The system according to the above (1), wherein the heat exchanger comprises a temperature sensor.
(21) The system according to the above (1), wherein the heat exchanger comprises a fan to circulate the fluid through the system.
(22) The system according to the above (21), wherein the fan is configured to operate at a variable rate.
(23) The system according to the above (21), wherein the fan is a first fan and the system further comprises at least a second fan.
(24) The system according to the above (1), wherein at least a portion of the recirculating fluid path is insulated.
(25) The system according to the above (1), wherein the recirculating fluid path comprises a temperature sensor.
(26) The system according to the above (2), wherein the recirculating fluid path comprises a guide vane configured to manage flow of the air.
(27) The system according to the above (1), wherein the generator further comprises a pump configured to circulate the fluid through the system.
(28) The system according to the above (1), further comprising insulation to insulate at least one component of the system.
(29) The system according to the above (1), further comprising a control system configured to control at least one system parameter.
(30) The system according to the above (1), further comprising a data acquisition module.
(31) The system according to the above (1), further comprising at least one sensor.
(32) The system according to the above (31), wherein the at least one sensor comprises at least one temperature sensor.
(33) The system according to the above (31), wherein the at least one sensor comprises at least one air flow sensor.
(34) The system according to the above (1), further comprising a nucleation triggering device.
(35) The system according to the above (34), wherein the nucleation triggering device comprises a valve.
(36) The system according to the above (1), further comprising a cassette configured to interface with the system.
(37) The system according to the above (36), wherein the cassette comprises a sterile loop configured for flow of the solution.
(38) The system according to the above (1), further comprising a delivery device configured to interface with the port.
(39) The system according to the above (38), wherein at least one portion of the delivery device is disposable.
(40) The system according to the above (38), wherein the delivery device comprises a cannula.
(41) The system according to the above (40), wherein the delivery device comprises a needle.
(42) The system according to the above (38), wherein the delivery device comprises a thermal jacket.
(43) The system according to the above (1), further comprising a container configured to interface with the repository.
(44) The system according to the above (43), wherein the container is disposable.
(45) The system according to the above (43), wherein the container comprises the solution.
(46) The system according to the above (1), wherein at least one component of the system comprises an identifier selected from the group consisting of a radio-frequency identification (RFID) tag, a chip, and a barcode.
(47) The system according to the above (1), wherein the heat exchanger is one of a plurality of heat exchangers.
(48) A method for generating a cold solution using a system comprising receiving a solution in the system; generating the cold solution from the solution; and transferring the cold solution from the system; wherein generating the cold solution comprises circulating a fluid.
(49) The method according to the above (48), wherein the fluid comprises air.
(50) The method according to the above (48), wherein generating the cold solution comprises cooling the fluid.
(51) The method according to the above (50), wherein the fluid is cooled to a temperature of about −10° C. to about −60° C.
(52) The method according to the above (50), wherein the fluid is cooled to a temperature of about −10° C. to about −40° C.
(53) The method according to the above (48), wherein generating the cold solution comprises cooling the solution.
(54) The method according to the above (48), wherein generating the cold solution comprises circulating the fluid through the system.
(55) The method according to the above (54), wherein the fluid is circulated through the system a variable rate.
(56) The method according to the above (49), wherein generating the cold solution comprises managing flow of the air.
(57) The method according to the above (48), wherein generating the cold solution comprises pumping the fluid through the system.
(58) The method according to the above (48), wherein transferring the cold solution comprises aseptic transfer.
(59) The method according to the above (48), further comprising controlling at least one system parameter.
(60) The method according to the above (48), further comprising acquiring data.
(61) The method according to the above (48), further comprising sensing an operating condition.
(62) The method according to the above (61), wherein the operating condition comprises temperature.
(63) The method according to the above (61), wherein the operating condition comprises air flow.
(64) The method according to the above (48), wherein generating the cold solution comprises triggering nucleation.
(65) The method according to the above (48), further comprising receiving a cassette configured to interface with the system.
(66) The method according to the above (65), further comprising causing the solution to flow in the cassette.
(67) The method according to the above (48), further comprising dispensing the cold solution into a delivery device via a port.
(68) The method according to the above (67), further comprising maintaining a temperature of the cold solution in the delivery device.
(69) The method according to the above (48), further comprising identifying at least one component of the system selected from the group consisting of a radio-frequency identification (RFID) tag, a chip, and a barcode.

### BRIEF DESCRIPTION OF DRAWINGS

FIG. 1 is a flowchart of a method according to an embodiment of the invention.

FIG. 2 depicts a system in accordance with certain embodiments.

FIG. 3 depicts part of a generator comprising at least one radiator.

FIG. 4 depicts part of a generator comprising an evaporator.

### DETAILED DESCRIPTION

Methods and systems for generating a cold solution are provided. In one application, the cold solution may be injected into subcutaneous fat of a subject to facilitate weight reduction or improve cosmetic appearances via cryolipolysis. Systems for generating a cold solution comprise a repository for receiving a solution, a generator for generating the cold solution from the solution, and a port for transferring the cold solution from the system. The generator comprises a cooling device, a heat exchanger, and a recirculating fluid path configured to circulate a fluid which may comprise air. Methods for generating a cold solution using a system comprise receiving a solution in the system, generating the cold solution from the solution, and transferring the cold solution from the system, wherein generating the cold solution comprises circulating a fluid which may comprise air.

Cold solution generators can be characterized by where a nucleation event occurs. Nucleation is the initial process at which ice crystals begin to form, and can be either on a surface, for example a surface of a system component, or in solution. Nucleation can be initiated, for example, via a nucleation triggering device such as a pinch valve, or nucleation can be spontaneous.

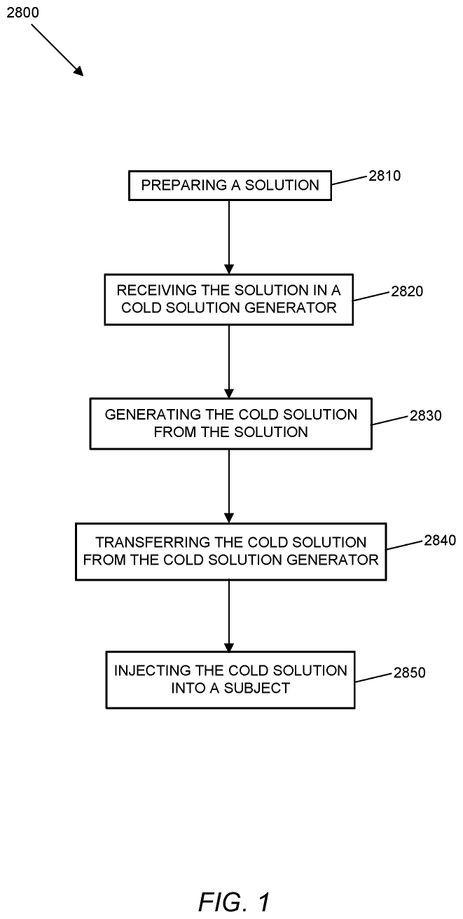

Methods of the invention are directed to generation of a cold solution. FIG. 1 is a flowchart of an exemplary embodiment of a method 2800 of the invention. The method 2800 comprises 2810 preparing a solution. Components of the solution, for example, liquid water and one or more additives, are selected to generate a cold solution with desired properties. The method 2800 further comprises 2820 receiving the solution in a cold solution generator. The solution may be inserted into the cold solution generator in any suitable manner. For example, the cold solution may be in a container and the container may be inserted into a repository of the cold solution generator. Alternatively, the solution can be provided directly to the cold solution generator via a port. The method 2800 further comprises 2830 generating the cold solution from the solution. Parameters of the cold solution generator are adjusted to generate the cold solution from the solution. For example, the temperature and flow rate are controlled to cool the solution and generate the cold solution. Once generated, the cold solution is ready for injection into a subject. The cold solution is transferred from the cold solution generator 2840 to a delivery device, for example, via a port for aseptic transfer.

The delivery device may be a cannula, such as a needle. The method 2800 further comprises 2850 injecting the cold solution into a subject. The cold solution may be injected by a healthcare professional in a manner consistent with a treatment plan, such as for injection into subcutaneous fat for fatremoval.

Embodiments of the invention are directed to systems for generating a cold solution. The systems comprise various attributes including but not limited to ease of dispensing of cold solution, sterile and disposable components of the system having fluid contact, time between required maintenance, size, set up time and ease, efficient use of solution, open/closed system, cool-down times, materials used, locations of cold extraction, mixing and maintenance of cold solution, volumes of cold solution generated, and level of control over various properties of the cold solution.

Systems for generating slurries additionally provide parameters and ranges that can be controlled and optimized. For example, each of the length of uncooled tubing, nucleation temperature, cold solution flow rate, cold solution dispensation rate, tubing geometry including tubing length, diameter, properties of surface materials, gas flow rate, temperature sensor positioning, and cold solution profiles including cooling temperature, maintenance temperature, and growth temperature can be varied and optimized.

By optimizing process parameters, the followed parameters can also be controlled and optimized including amount of usable cold solution, stratification of crystals, degree of ice coagulation, ice growth rate, ice fraction, crystal size, crystal shape and smoothing, ingress of particulates, amount of air entrained, maintenance period, and particulate generation.

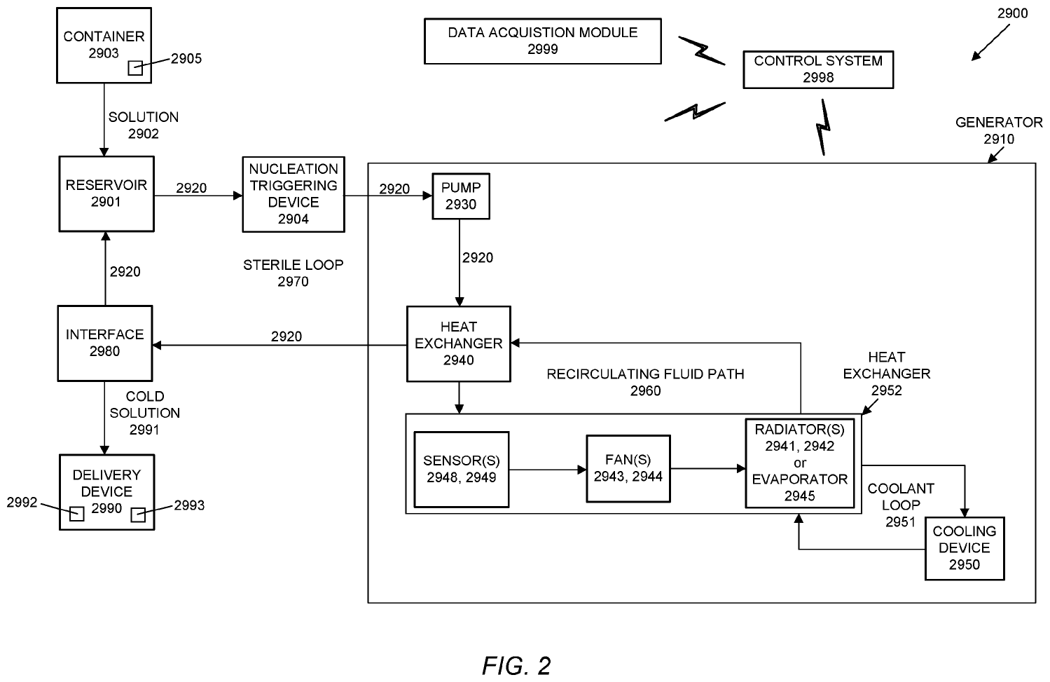

FIG. 2 shows an embodiment of a system 2900 for generating a cold solution 2991.

The sterile loop 2970 is configured for receiving a solution 2902. In some aspects, such as via a reservoir 2901 configured to interface with a solution container 2903 which may be disposable. The solution 2902 is pumped into a generator 2910 for generating the cold solution 2991 from the solution 2902 through tubing 2920 by a pump 2930, which can be any suitable pump such as a peristaltic pump. Any suitable tubing may be used. Tubing may include any or all of a tube and/or a molded or extruded flow path. Tubing may comprise a slight curvature (or other suitable configuration) to break up the ice as it is dispensed. In some embodiments, the solution is cooled using at least one heat exchanger 2940 and/or 2952 and a cooling device 2950.

The system 2900 includes a sterile loop 2970 configured for flow of the solution 2902. The sterile loop 2970 connects at least the reservoir 2901 and the heat exchanger 2940. In some variations, the sterile loop 2970 may also connect to the pump 2930, a nucleation triggering device 2904, and an interface 2980. For example, the solution enters the sterile loop 2970 and is continually cooled down and circulated during cold solution generation. The generator 2910 may include a recirculating fluid path 2960 configured to circulate a fluid. The fluid may comprise air. Accordingly, the clinical risk of contamination of the solution 2902 with liquid coolant is avoided. The recirculating fluid path 2960 connects at least the heat exchanger 2940 and the heat exchanger 2952 which includes either: (i) at least one radiator 2941 or 2942; or (ii) an evaporator 2945. The sterile loop 2970 may interface with the fluid in the recirculating fluid path 2960 via the heat exchanger 2940. In variations not shown, heat exchanger 2940 may be omitted and the sterile loop 2970 may interface with the fluid in the recirculating fluid path 2960 via the heat exchanger 2952.

The generator 2910 may include a coolant loop 2951 configured for flow of a coolant such as a single-phase coolant (e.g., a liquid coolant for use with the at least one radiator 2941 or 2942 as shown in FIGS. 2 & 3) or a multi-phase refrigerant (e.g., for use with the evaporator 2945 as shown in FIGS. 2 & 4). The coolant loop 2951 connects at least the cooling device 2950 and either: (i) the at least one radiator 2941 or 2942; or (ii) the evaporator 2945. The cooling device 2950 may interface with the heat exchanger 2952 via either: (i) the at least one radiator 2941 or 2942 (as shown in FIGS. 2 & 3); or (ii) the evaporator 2945 (as shown in FIGS. 2 & 4).

The coolant loop 2951 and the sterile loop tubing 2920 are remote from one another such that the coolant loop 2951 does not come into contact with the sterile loop tubing 2920. Accordingly, the clinical risk of contamination of the solution 2902 with liquid coolant is avoided.

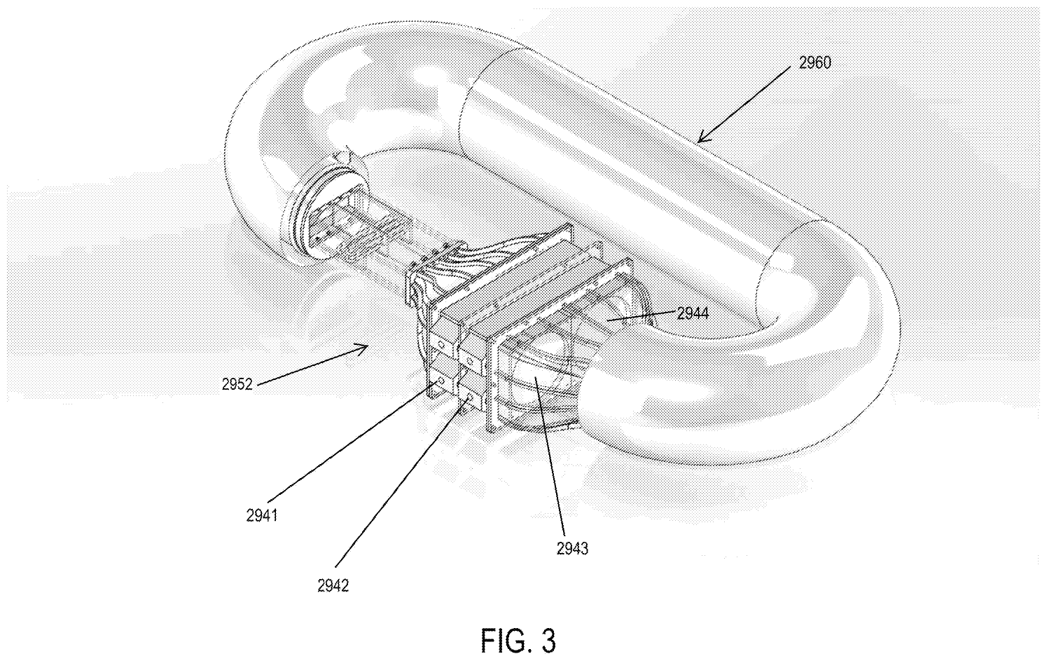

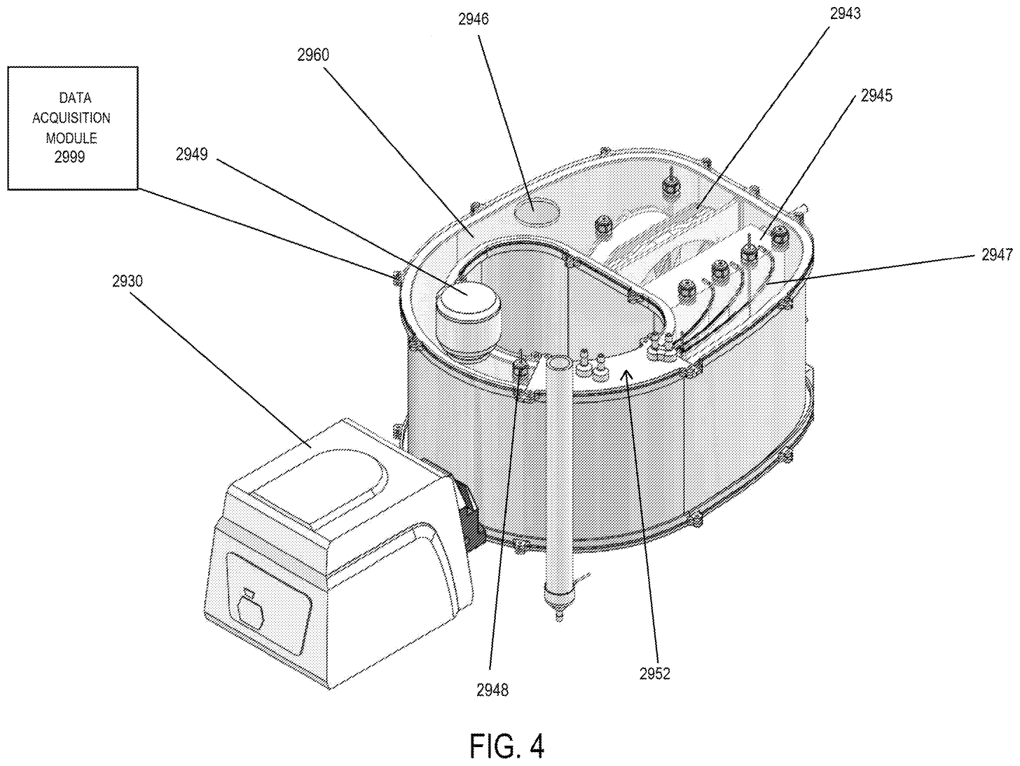

Either heat exchanger 2940 or 2952 may include a diffuser and/or a reducer. Components of either heat exchanger 2940 or 2952 may be comprised of plastics, silicone, and/or may be three-dimensionally (3D) printed such as Fused Deposition Modeling (FDM) 3D printed. For example, components of either heat exchanger 2940 or 2952 may comprise high-density polyethylene (HDPE). The cooling device 2950 may be configured as, for example, a chiller (e.g., as shown in FIG. 3) and/or a vapor compression cooling system (e.g., as shown in FIG. 4). The cooling device 2950 may include a heat pump, a compressor and/or a condenser. The cooling device 2950 may be configured to operate at a temperature of about −10° C. to about −60° C., for example, at a temperature of about −10° C. to about −40° C. Accordingly, the fluid may be cooled to a temperature of about −10° C. to about −60° C., for example, at a temperature of about −10° C. to about −40° C.

Once cold solution generation is complete, the cold solution 2991 may be transferred. The generator 2910 includes an interface 2980 for transferring the solution 2902 from the heat exchanger 2940 back to the reservoir 2901 and/or transferring the cold solution 2991 from the system to, for example, a delivery device 2990. The interface 2980 may comprise, for example, a port and/or a Y-joint. The delivery device 2990 may comprise one or more of a thermal jacket 2992, and a cannula such as a needle 2993. At least one portion of the delivery device 2990 may be disposable. A temperature of the cold solution 2991 in the delivery device 2990 may be maintained (e.g., at a temperature at or below 0° C.).

The cold solution generation system is effectively a completely closed system, allowing for sterility to be maintained while drawing the cold solution from the system for injection.

The system 2900 may also include nucleation triggering device 2904 connected to the circulating system for nucleating the solution such that ice particle formation is initiated. In some embodiments, the nucleation triggering device may include a valve. In some embodiments, nucleation occurs spontaneously.

The system 2900 may further include valves for liquids or gases to be input or removed from the reservoir 2901, such as a valve to allow the cold solution 2991 to be removed from the reservoir 2901, a valve to allow cold solution or a solution to enter the reservoir 2901, and/or a cleaning valve to input a gas or sterilizing solution to clean the cold solution reservoir 2901 and/or valves, among other components.

The system 2900 may include insulation to insulate at least one component of the system 2900. For example, any or all of the reservoir 2901, the interface 2980, any or all of the sterile loop 2970, any or all of the cooling device 2950, any or all of the coolant loop 2951, and/or any or all of the recirculating fluid path 2960 may include insulation. In some variations, the insulation may be any of polystyrene insulation, foam insulation and/or bubble-foil insulation.

In some embodiments, the container 2903 comprises a container identifier 2905. In some embodiments, the container identifier 2905 is selected from the group consisting of a radio-frequency identification (RFID) tag, a chip, and a barcode.

Elements of the system 2900 may be included in a cassette configured to interface with the system 2900. For example, the sterile loop 2970 may be configured as part of the cassette which can be removed and reinserted into the system 2900. Accordingly, cold air may be circulated through the cassette while the sterile loop 2970 is protected within the cassette. In some variations, any of the reservoir 2901, the nucleation triggering device 2904, the tubing 2920, the pump 2930, the heat exchanger 2940 and/or the interface 2980 may be included in the cassette. The cassette may be single-use or disposable and may have various form factors.

A control system 2998 controls at least one system parameter of the pump 2930, the heat exchanger 2940, the cooling device 2950, the heat exchanger 2952, the recirculating fluid path 2960, and/or the sterile loop 2970 such as flow rate (e.g., 10 mL/min), pressure, and/or temperature. The controller 2998 communicates with the system components but may be internal to the system 2900 or external to the system 2900. A data acquisition module 2999 may be provided separately for acquiring data which may relate to one or more system parameters of the system 2900. The data acquisition module 2999 communicates with the control system 2998 but may be internal to the control system 2998 or external to the control system 2998.

FIG. 3 depicts part of generator 2910 in accordance with FIG. 2 wherein the generator 2910 includes at least one radiator 2941 or 2942 (instead of an evaporator 2945). As shown in this embodiment, generator 2910 is provided with a heat exchanger 2952 which may include at least one radiator 2941 or 2942. In some variations, the heat exchanger 2952 includes at least two radiators 2941 and 2942. Accordingly, flow of the fluid may be split between the two radiators 2941 and 2942 as the fluid flows in parallel through both radiators 2941 and 2942. The heat exchanger 2952 may include at least one fan 2943 or 2944 to circulate the fluid through the recirculating fluid path 2960 back to the heat exchanger 2952. In some variations, the heat exchanger 2952 includes at least two fans 2943 and 2944. Any or all of the fans 2943 and 2944 may be configured to operate at a variable rate. The heat exchanger 2952 interfaces with the cooling device 2950 via the at least one radiator 2941 or 2942.

The heat exchanger 2952 may further comprise at least one sensor 2948 or 2949 as shown in FIG. 2. In some variations, the heat exchanger 2952 includes a temperature sensor 2948. The temperature sensor 2948 may be, for example, a resistance temperature detector (RTD). The heat exchanger 2952 may further comprise an air flow sensor 2949. The air flow sensor 2949 may be, for example, a hot-wire anemometer (HWA). In addition or alternative to the heat exchanger 2952, the system 2900 can include sensors at any suitable location such as in one or more locations in the sterile loop 2970 and/or the coolant loop 2951.

The coolant loop 2951 and the sterile loop tubing 2920 are remote from one another such that the coolant loop 2951 does not come into contact with the sterile loop tubing 2920. Accordingly, the clinical risk of contamination of the solution 2902 with liquid coolant is avoided.

FIG. 4 depicts part of generator 2910 in accordance with FIG. 2 wherein the generator 2910 includes an evaporator 2945 (instead of at least one radiator 2941 or 2942). The heat exchanger 2952 may include a fan 2943 to circulate the fluid through the recirculating fluid path 2960 back to the heat exchanger 2952. The fan 2943 may be configured to operate at a variable rate. The fan 2943 may be an axial fan. The heat exchanger 2952 interfaces with the cooling device 2950 via the evaporator 2945. As shown in this embodiment, the generator 2910 includes the pump 2930, the heat exchanger 2952 and the evaporator 2945. An access port 2946 for the recirculating fluid path 2960 may be provided. As noted above, the heat exchanger 2952 interfaces with the cooling device 2950 via the evaporator 2945. The recirculating fluid path 2960 may comprise at least one guide vane 2947 configured to manage flow of the air. The at least one guide vane 2947 may comprise a curvature (or other suitable configuration) to maximize local air speed and minimize flow separation. The recirculating fluid path 2960 may be defined between an internal chassis and an external chassis.

The heat exchanger 2952 may further comprise at least one sensor 2948 or 2949. In some variations, the heat exchanger 2952 includes a temperature sensor 2948. The temperature sensor 2952 may be, for example, a resistance temperature detector (RTD). The heat exchanger 2952 may further comprise an air flow sensor 2949. The air flow sensor 2949 may be, for example, a hot-wire anemometer (HWA). In addition or alternative to the heat exchanger 2952, the system 2900 can include sensors at any suitable location such as in one or more locations in the sterile loop 2970 and/or the coolant loop 2951.

The coolant loop 2951 and the sterile loop tubing 2920 are remote from one another such that the coolant loop 2951 does not come into contact with the sterile loop tubing 2920. Accordingly, the clinical risk of contamination of the solution with the multi-phase refrigerant is avoided.

Aspects of the invention described herein, such as monitoring and controlling of various parameters, can be performed using any type of computing device, such as a computer or programmable logic controller (PLC), that includes a processor, e.g., a central processing unit, or any combination of computing devices where each device performs at least part of the process or method. In some embodiments, systems and methods described herein may be performed with a handheld device, e.g., a smart tablet, a smart phone, or a specialty device produced for the system.

Methods of the present disclosure can be performed using software, hardware, firmware, hardwiring, or combinations of any of these. Features implementing functions can also be physically located at various positions, including being distributed such that portions of functions are implemented at different physical locations (e.g., cold solution generator in one room and host workstation in another, or in separate buildings, for example, with wireless or wired connections).

Processors suitable for the execution of computer programs include, by way of example, both general and special purpose microprocessors, and any one or more processor of any kind of digital computer. Generally, a processor will receive instructions and data from a read-only memory or a random access memory or both. Elements of computer are a processor for executing instructions and one or more memory devices for storing instructions and data.

Generally, a computer will also include, or be operatively coupled to receive data from or transfer data to, or both, one or more non-transitory mass storage devices for storing data, e.g., magnetic, magneto-optical disks, or optical disks. In some embodiments, sensors on the system send process data via Bluetooth to a central data collection unit located outside of an incubator. In some embodiments, data is sent directly to the cloud rather than to physical storage devices. Information carriers suitable for embodying computer program instructions and data include all forms of non-volatile memory, including by way of example semiconductor memory devices, (e.g., EPROM, EEPROM, solid state drive (SSD), and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an input or output (I/O) device, e.g., a CRT, LCD, LED, or projection device for displaying information to the user and an I/O device such as a keyboard and a pointing device, (e.g., a mouse or a trackball), by which the user can provide input to the computer. Other kinds of devices can be used to provide for interaction with a user as well. For example, feedback provided to the user can be any form of sensory feedback (e.g., visual feedback, auditory feedback, or tactile feedback), and input from the user can be received in any form including acoustic, speech, or tactile input.

The subject matter described herein can be implemented in a computing system that includes a back-end component (e.g., a data server), a middleware component (e.g., an application server), or a front-end component (e.g., a client computer having a graphical user interface or a web browser through which a user can interact with an implementation of the subject matter described herein), or any combination of such back-end, middleware, and front-end components. The components of the system can be interconnected through a network by any form or medium of digital data communication, e.g., a communication network. Examples of a communication network include a cell network (e.g., 3G, 4G or 5G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, data processing apparatus (e.g., a programmable processor, a computer, or multiple computers). A computer program (also known as a program, software, software application, app, macro, or code) can be written in any form of programming language, including compiled or interpreted languages (e.g., C, C++, Perl), and it can be deployed in any form, including as a stand-alone program or as a module, component, subroutine, or other unit suitable for use in a computing environment. Systems and methods of the invention can include instructions written in any suitable programming language known in the art, including, without limitation, C, C++, Perl, Java, ActiveX, HTML5, Visual Basic, or JavaScript.

A computer program does not necessarily correspond to a file. A program can be stored in a file or a portion of file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over a network (e.g., as packets being sent from a server to a client, for example, through a Network Interface Card, modem, wireless card, or similar).

Writing a file according to embodiments of the invention involves transforming a tangible, non-transitory, computer-readable medium, for example, by adding, removing, or rearranging particles (e.g., with a net charge or dipole moment into patterns of magnetization by read/write heads), the patterns then representing new collocations of information about objective physical phenomena desired by, and useful to, the user. In some embodiments, writing involves a physical transformation of material in tangible, non-transitory computer readable media (e.g., with certain optical properties so that optical read/write devices can then read the new and useful collocation of information, e.g., burning a CD-ROM). In some embodiments, writing a file includes transforming a physical flash memory apparatus such as NAND flash memory device and storing information by transforming physical elements in an array of memory cells made from floating-gate transistors. Methods of writing a file are well-known in the art and, for example, can be invoked manually or automatically by a program or by a save command from software or a write command from a programming language.

Suitable computing devices typically include mass memory, at least one graphical user interface, at least one display device, and typically include communication between devices. The mass memory illustrates a type of computer-readable media, namely computer storage media. Computer storage media may include volatile, nonvolatile, removable, and non-removable media implemented in any method or technology for storage of information, such as computer readable instructions, data structures, program modules, or other data. Examples of computer storage media include RAM, ROM, EEPROM, flash memory, or other memory technology, CD-ROM, digital versatile disks (DVD) or other optical storage, magnetic cassettes, magnetic tape, magnetic disk storage or other magnetic storage devices, RFID tags or chips, or any other medium which can be used to store the desired information and which can be accessed by a computing device.

As one skilled in the art would recognize as necessary or best-suited for performance of the methods of the invention, a computer system or machines employed in embodiments of the invention may include one or more processors (e.g., a central processing unit (CPU) a graphics processing unit (GPU) or both), a main memory and a static memory, which communicate with each other via a bus.

In certain embodiments, the cold solution generation system is aseptically assembled using terminally sterilized components. The cold solution generation system is effectively a completely closed system, allowing for sterility to be maintained while continually drawing the cold solution from the system for injection.

Since the cold solution is to be injected into humans, several sterility and quality requirements exist for the systems. Sterility may be confirmed by performing any suitable validation tests.

The cold solution is deliverable to a subject via injection. The cold solution may be delivered by any suitable delivery device, such as a cannula, for example, a syringe. The syringe can be formed from any type of biocompatible, pharmacologically inert material suitable for coming in contact with fluids to be provided within a human body. In order to pass through the needle of a syringe without getting stuck or blocking flow of the cold solution, the largest cross-section of the ice particles must be smaller than the internal diameter of the needle used for injection.

In view of the above, the solution can be cleanly and effectively cooled by air and the clinical risk of potential contamination of the solution associated with available methods using a liquid coolant can be avoided.

The generators of the present invention may be used to generate a cold solution, for example, a slurry as described in international application serial number PCT/US19/54828, filed on Oct. 4, 2019, which is incorporated by reference in its entirety herein.

In some aspects, the cold solution may include water. In some aspects, the cold solution may include water and one or more additives. In some embodiments, the one or more additives are inactive, biocompatible ingredients, including any substance (at or below their respective indicated concentrations) in the FDA GRAS list, which is incorporated by reference in its entirety herein. In some embodiments, the additives can comprise one or more of a salt, a sugar, and a thickener.

In some embodiments, the cold solution can comprise a temperature range of about −25° C. to about 10° C., for example, about −25, −24, −23, −22, −21, −20, −19, −18, −17, −16, −15, −14, −13, −12, −11, −10, −9, −8, −7, −6, −5, −4, −3, −2, −1, 0, 1, 2, 3, 4, 5, 6, 7, 8, 9 or 10° C.

In some aspects, the cold solution comprises potassium chloride at about 0.02% by mass or lower, for example, 0.19, 0.18, 0.17, 0.16, 0.15, 0.14, 0.13, 0.12, 0.11, 0.10, 0.09, 0.08, 0.07, 0.06, 0.05, 0.04, 0.03, 0.02, 0.01, or 0% by mass. In some aspects, the cold solution comprises calcium chloride at about 0.02% by mass or lower, for example, 0.19, 0.18, 0.17, 0.16, 0.15, 0.14, 0.13, 0.12, 0.11, 0.10, 0.09, 0.08, 0.07, 0.06, 0.05, 0.04, 0.03, 0.02, 0.01, or 0% by mass. In some aspects, the cold solution comprises sodium chloride at about 2.25% by mass or lower, for example at about 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1, 0.05 or 0% by mass. In some aspects, the cold solution comprises magnesium chloride at about 0.02% by mass or lower, for example, 0.19, 0.18, 0.17, 0.16, 0.15, 0.14, 0.13, 0.12, 0.11, 0.10, 0.09, 0.08, 0.07, 0.06, 0.05, 0.04, 0.03, 0.02, 0.01, or 0% by mass.

In some aspects, the cold solution comprises sucrose at about 5% by mass or lower, for example at about 5, 4.5, 4, 3.5, 3, 2.5, 2, 1.5, 1, 0.5, or 0% by mass. In some aspects, the cold solution comprises dextrose at about 5.6% by mass or lower, for example at about 5.5, 5, 4.5, 4, 3.5, 3, 2.5, 2, 1.5, 1, 0.5, or 0% by mass. In some aspects, the cold solution comprises mannitol at about 4.95% by mass or lower, for example at about 4.5, 4, 3.5, 3, 2.5, 2, 1.5, 1, 0.5, or 0% by mass. In some aspects, the cold solution comprises lactose at about 0.45% by mass or lower, for example at about 0.4, 0.35, 0.3, 0.25, 0.2, 0.15, 0.1, 0.05, or 0% by mass. In some aspects, the cold solution comprises sorbitol at about 4.7% by mass or lower, for example at about 4.5, 4, 3.5, 3, 2.5, 2, 1.5, 1, 0.5, or 0% by mass. In some aspects, the cold solution comprises glycerol at about 2% by mass or lower, for example at about 1.9, 1.8, 1,7, 1.6, 1.5, 1.4, 1.3, 1.2, 1.1, 1.0, 0.9, 0.8, 0.7, 0.6, 0.5, 0.4, 0.3, 0.2, 0.1, 0.05 or 0% by mass.

In some aspects, the cold solution comprises hetastarch at about 6% by mass or lower, for example at about 5.5, 5, 4.5, 4, 3.5, 3, 2.5, 2, 1.5, 1, 0.5, or 0% by mass. In some aspects, the cold solution comprises pectin at about 16.7% by mass or lower, for example at about 16, 15, 10, 9, 8, 7, 6, 5, 4, 3, 2, 1 or 0% by mass. In some aspects, the cold solution comprises polyethylene glycol at about 20% by mass or lower, for example at about 20, 15, 10, 9, 8, 7, 6, 5, 4, 3, 2, 1 or 0% by mass. In some aspects, the cold solution comprises gelatin at about 16% by mass or lower, for example at about 15, 10, 9, 8, 7, 6, 5, 4, 3, 2, 1 or 0% by mass. In some aspects, the cold solution comprises sodium methylcellulose at about 5% by mass or lower, for example at about 5, 4.5, 4, 3.5, 3, 2.5, 2, 1.5, 1, 0.5, or 0% by mass. In some aspects, the cold solution comprises a sodium alginate at about 5% by mass or lower, for example at about 5, 4.5, 4, 3.5, 3, 2.5, 2, 1.5, 1, 0.5, or 0% by mass. In some aspects, the cold solution comprises polyvinyl alcohol at about 5% by mass or lower, for example at about 5, 4.5, 4, 3.5, 3, 2.5, 2, 1.5, 1, 0.5, or 0% by mass. In some aspects, the cold solution comprises polyvinyl pyrrolidone (PVP) at about 5% by mass or lower, for example at about 5, 4.5, 4, 3.5, 3, 2.5, 2, 1.5, 1, 0.5, or 0% by mass. In some aspects, the cold solution comprises Xanthan Gum at about 0.75% by mass or lower, for example at about 0.7, 0.6, 0.5, 0.4, 0.3, 0.2, 0.1, 0.05 or 0% by mass. In some aspects, the cold solution comprises CMC at about 0.75% by mass or lower, for example at about 0.7, 0.6, 0.5, 0.4, 0.3, 0.2, 0.1, 0.05 or 0% by mass. In some aspects, the cold solution comprises guar gum at about 1% by mass or lower, for example at about 0.9, 0.8, 0.7, 0.6, 0.5, 0.4, 0.3, 0.2, 0.1, 0.05 or 0% by mass. In some aspects, the cold solution comprises locust bean gum at about 1% by mass or lower, for example at about 0.9, 0.8, 0.7, 0.6, 0.5, 0.4, 0.3, 0.2, 0.1, 0.05 or 0% by mass. In some aspects, the cold solution comprises gum tracanth at about 1% by mass or lower, for example at about 0.9, 0.8, 0.7, 0.6, 0.5, 0.4, 0.3, 0.2, 0.1, 0.05 or 0% by mass. In some aspects, the cold solution comprises carbomer at about 1% by mass or lower, for example at about 0.9, 0.8, 0.7, 0.6, 0.5, 0.4, 0.3, 0.2, 0.1, 0.05 or 0% by mass.

In some aspects of the invention, for intradermal, subcutaneous, or intramuscular routes of administration, additives may include sodium chloride, saline, glycerin/glycerol, dextrose, sodium CMC, xanthan gum, and polyethylene glycol. For example, acceptable concentrations of saline are about 0.9% for soft tissue use and about 2.25% for subcutaneous use, while acceptable concentrations of glycerin/glycerol are about 1.6% to about 2.0% for dermal use and about 15% for subcutaneous use. For example, acceptable concentrations of dextrose are about 5% w/v for intramuscular use and about 7.5% per unit dose for intramuscular-subcutaneous use. For example, acceptable concentrations of sodium CMC are about 0.75% for intralesional use, about 3% for intramuscular use, and about 0.5% to about 0.75% for soft tissue use. As another example, acceptable concentrations of xanthan gum are about 1% for intra-articular use in animal studies and about 0.6% for FDA ophthalmic use. For example, acceptable concentrations of polyethylene glycol, such as Polyethylene Glycol 3350, are about 2.0% to about 3.0% for FDA soft tissue use and about 4.42% for subcutaneous use.

In certain aspects, the cold solution has an osmolarity lower than about 2,200 mOsm/L. In some aspects, the osmolarity is less than about 1,000 mOsm/L. In some aspects, the osmolarity is less than about 600 mOsm/L. In such an aspect, the cold solution may comprise about 0.9% saline; about 1.0% to about 2.0% dextrose; about 1.0% to about 1.6% glycerol; less than about 0.5% sodium CMC; and less than about 0.6% xanthan gum. In one aspect, the cold solution may be about 500 mOsm/kg to about 700 mOsm/kg and comprise about 0.9% to about 1.4% saline; about 2.0% to about 4.0% dextrose; about 1.7% to about 2.0% glycerol; about 0.6% to about 1.0% sodium CMC; and about 0.6% to about 1.0% xanthan gum. In another aspect, the cold solution composition may be about 700 mOsm/kg to about 900 mOsm/kg and comprise about 1.5% to about 1.7% saline; about 5.0% to about 7.5% dextrose; about 3.0% to about 5.0% glycerol; about 1.0% to about 3.0% sodium CMC; and about 1.0% xanthan gum. In some aspects, the cold solution composition may be greater than about 1,000 mOsm/kg. In such an aspect, the cold solution may comprise about 1.8% to about 3.0% saline; about 10% dextrose; greater than about 5.0% glycerol; sodium CMC; and xanthan gum. In another aspect, the cold solution can be isotonic relative to the subject's cells, e.g., having an osmolarity of about 308 mOsm/kg. In such an aspect, the cold solution may include normal saline and 2% glycerol. Additives can be selected and included in any concentration suitable to generate a cold solution have certain characteristics, for example to increase or decrease the osmolality.

Unless specifically stated or obvious from context, as used herein, the term “about” is understood as within a range of normal tolerance in the art, for example, within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term “about”.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What we claim is:

1. A system for generating a cold solution, the system comprising:
a repository for receiving a solution;
a generator for generating the cold solution from the solution; and
a port for transferring the cold solution from the system;
wherein the generator comprises a cooling device, a heat exchanger, and a recirculating fluid path configured to circulate a fluid.

2. The system of claim 1, wherein the fluid comprises air.

3. The system of claim 1, wherein the repository is insulated.

4. The system of claim 1, further comprising a valve configured to control flow.

5. The system of claim 1, further comprising a sterile loop configured for flow of the solution.

6. The system of claim 5, wherein the sterile loop interfaces with the fluid in the recirculating fluid path via the heat exchanger.

7. The system of claim 1, wherein the port is insulated.

8. The system of claim 1, wherein the port is configured for aseptic transfer.

9. The system of claim 8, further comprising a valve configured to control flow.

10. The system of claim 1, wherein the cooling device is configured as a cold sink.

11. The system of claim 10, wherein the cooling device is configured to operate at a temperature of about −10° C. to about −60° C.

12. The system of claim 10, wherein the cooling device is configured to operate at a temperature of about −10° C. to about −40° C.

13. The system of claim 1, wherein the cooling device comprises a single-phase coolant.

14. The system of claim 1, wherein the cooling device comprises a multi-phase refrigerant.

15. The system of claim 1, wherein the cooling device interfaces with the heat exchanger.

16. The system of claim 1, wherein the heat exchanger comprises a radiator.

17. The system of claim 16, wherein the heat exchanger interfaces with the cooling device via the radiator.

18. The system of claim 1, wherein the heat exchanger comprises an evaporator.

19. The system of claim 18, wherein the heat exchanger interfaces with the cooling device via the evaporator.

20. The system of claim 1, wherein the heat exchanger comprises a temperature sensor.

21. The system of claim 1, wherein the heat exchanger comprises a fan to circulate the fluid through the system.

22. The system of claim 21, wherein the fan is configured to operate at a variable rate.

23. The system of claim 21, wherein the fan is a first fan and the system further comprises at least a second fan.

24. The system of claim 1, wherein at least a portion of the recirculating fluid path is insulated.

25. The system of claim 1, wherein the recirculating fluid path comprises a temperature sensor.

26. The system of claim 2, wherein the recirculating fluid path comprises a guide vane configured to manage flow of the air.

27. The system of claim 1, wherein the generator further comprises a pump configured to circulate the fluid through the system.

28. The system of claim 1, further comprising insulation to insulate at least one component of the system.

29. The system of claim 1, further comprising a control system configured to control at least one system parameter.

30. The system of claim 1, further comprising a data acquisition module.

31. The system of claim 1, further comprising at least one sensor.

32. The system of claim 31, wherein the at least one sensor comprises at least one temperature sensor.

33. The system of claim 31, wherein the at least one sensor comprises at least one air flow sensor.

34. The system of claim 1, further comprising a nucleation triggering device.

35. The system of claim 34, wherein the nucleation triggering device comprises a valve.

36. The system of claim 1, further comprising a cassette configured to interface with the system.

37. The system of claim 36, wherein the cassette comprises a sterile loop configured for flow of the solution.

38. The system of claim 1, further comprising a delivery device configured to interface with the port.

39. The system of claim 38, wherein at least one portion of the delivery device is disposable.

40. The system of claim 38, wherein the delivery device comprises a cannula.

41. The system of claim 40, wherein the delivery device comprises a needle.

42. The system of claim 38, wherein the delivery device comprises a thermal jacket.

43. The system of claim 1, further comprising a container configured to interface with the repository.

44. The system of claim 43, wherein the container is disposable.

45. The system of claim 43, wherein the container comprises the solution.

46. The system of claim 1, wherein at least one component of the system comprises an identifier selected from the group consisting of a radio-frequency identification (RFID) tag, a chip, and a barcode.

47. The system of claim 1, wherein the heat exchanger is one of a plurality of heat exchangers.

48. A method for generating a cold solution using a system, the method comprising:
receiving a solution in the system;
generating the cold solution from the solution; and
transferring the cold solution from the system;
wherein generating the cold solution comprises circulating a fluid.

49. The method of claim 48, wherein the fluid comprises air.

50. The method of claim 48, wherein generating the cold solution comprises cooling the fluid.

51. The method of claim 50, wherein the fluid is cooled to a temperature of about −10° C. to about −60° C.

52. The method of claim 50, wherein the fluid is cooled to a temperature of about −10° C. to about −40° C.

53. The method of claim 48, wherein generating the cold solution comprises cooling the solution.

54. The method of claim 48, wherein generating the cold solution comprises circulating the fluid through the system.

55. The method of claim 54, wherein the fluid is circulated through the system a variable rate.

56. The method of claim 49, wherein generating the cold solution comprises managing flow of the air.

57. The method of claim 48, wherein generating the cold solution comprises pumping the fluid through the system.

58. The method of claim 48, wherein transferring the cold solution comprises aseptic transfer.

59. The method of claim 48, further comprising controlling at least one system parameter.

60. The method of claim 48, further comprising acquiring data.

61. The method of claim 48, further comprising sensing an operating condition.

62. The method of claim 61, wherein the operating condition comprises temperature.

63. The method of claim 61, wherein the operating condition comprises air flow.

64. The method of claim 48, wherein generating the cold solution comprises triggering nucleation.

65. The method of claim 48, further comprising receiving a cassette configured to interface with the system.

66. The method of claim 65, further comprising causing the solution to flow in the cassette.

67. The method of claim 48, further comprising dispensing the cold solution into a delivery device via a port.

68. The method of claim 67, further comprising maintaining a temperature of the cold solution in the delivery device.

69. The method of claim 48, further comprising identifying at least one component of the system selected from the group consisting of a radio-frequency identification (RFID) tag, a chip, and a barcode.
